(19) United States
(12) Patent Application Publication
(10) Pub. No.: US 2014/0276452 A1
(43) Pub. Date: Sep. 18, 2014

(54) FLUID EFFICIENT SPIKE

(71) Applicant: MEDRAD, INC., Indianola, PA (US)

(72) Inventors: Kevin P. COWAN, Allison Park, PA (US); Raymond C. HOFFMAN, Gibsonia, PA (US)

(73) Assignee: MEDRAD, INC., Indianola, PA (US)

(21) Appl. No.: 13/799,169

(22) Filed: Mar. 13, 2013

(51) Int. Cl. A61M 39/10; A61M 5/14

(52) U.S. Cl. CPC A61M 39/10; A61M 5/14
USPC 604/244

(57) ABSTRACT

Spikes, spike and tubing sets and fluid administrations systems are disclosed. A fluid administration system may include a fluid container having a septum and containing a fluid, a spike, and a flexible tube. The spike may include a hollow access tube extending from a proximal end to a distal end of the spike, a platform surrounding the hollow access tube at a location between the distal and proximal ends, and a sharp tip-end at the distal end. The access tube has a central bore and a sidewall, each extending from the proximal to the distal end. The access tube may include at least one opening extending from the central bore to the sidewall and located between the platform and the sharp tip-end. The flexible tube is configured to connect to the access tube and to deliver fluid from the access tube to a fluid administration location.

### BACKGROUND

Medicinal fluids or solutions intended for patient administration are frequently provided in an inverted bag having a septum or other access port capable of being pierced on the bottom of the bag. A sharp hollow spike attached to a tubing set may be introduced through the septum, thus allowing the bag contents to be passed via gravity through the tubing set. A conventional spike is hollow throughout and allows a fluid or solution to flow through a tip-end of the sharp hollow spike from the drip bag. The fluid or solution will continue to flow until the meniscus of the fluid/solution in the drip bag falls below the tip-end of the spike, leaving some residual fluid/solution in the bag.

Gravity-fed access to fluids may be used for other containers as well. For example, glass vials with a rubber septum covering may employ gravity-fed access. A spike and tubing set may provide access to a fluid from an inverted septum-covered vial as well. While drip bags typically contain a relatively large volume of fluid (such as 500 mL to 1000 mL), glass vial containers may include significantly less material (such as 10 mL or less). Moreover, fluids or solutions supplied in containers containing such small quantities tend to be expensive. As a result, fluid waste may result in a significant cost.

### SUMMARY

The invention described in this document is not limited to the particular systems, methodologies or protocols described, as these may vary. The terminology used herein is for the purpose of describing particular embodiments only, and is not intended to limit the scope of the present disclosure.

It must be noted that as used herein and in the appended claims, the singular forms “a,” “an,” and “the” include plural reference unless the context clearly dictates otherwise. Unless defined otherwise, all technical and scientific terms used herein have the same meanings as commonly understood by one of ordinary skill in the art. As used herein, the term “comprising” means “including, but not limited to.”

In an embodiment, a spike for fluid administration may include a hollow access tube extending from a proximal end of the spike to a distal end of the spike, a platform surrounding the hollow access tube at a location between the distal end and the proximal end, and a sharp tip-end located at the distal end of the spike. The hollow access tube has a central bore and a sidewall. Each of the central bore and the sidewall may extend from the proximal end to the sharp tip-end. The hollow access tube may include at least one opening extending from the central bore to the sidewall and located between the platform and the sharp tip-end.

In an embodiment, a spike and tubing set for fluid administration may include a spike and a flexible tube. The spike may include a hollow access tube extending from a proximal end of the spike to a distal end of the spike, a platform surrounding the hollow access tube at a location between the distal end and the proximal end, and a sharp tip-end located at the distal end of the spike. The hollow access tube has a central bore and a sidewall. Each of the central bore and the sidewall may extend from the proximal end to the sharp tip-end. The hollow access tube may include at least one opening extending from the central bore to the sidewall and located between the platform and the sharp tip-end. The flexible tube may be configured to connect to the hollow access tube and to deliver fluid from the hollow access tube to a fluid administration location.

In an embodiment, a fluid administration system may include a fluid container, a spike, and a flexible tube. The fluid container may include a septum and may contain a fluid. The spike may include a hollow access tube extending from a proximal end of the spike to a distal end of the spike, a sealing surface surrounding the hollow access tube at a location between the distal end and the proximal end, and a sharp tip-end located at the distal end of the spike. The hollow access tube has a central bore and a sidewall. Each of the central bore and the sidewall may extend from the proximal end to the sharp tip-end. The hollow access tube may include at least one opening extending from the central bore to the sidewall and located between the sealing surface and the sharp tip-end. The flexible tube may be configured to connect to the hollow access tube and to deliver fluid from the hollow access tube to a fluid administration location.

### BRIEF DESCRIPTION OF THE DRAWINGS

FIGS. 1-3 depict a plurality of illustrative spikes for fluid administration according to various embodiments.

FIG. 4 depicts an illustrative fluid administration system according to an embodiment.

FIG. 5A depicts an illustrative spike for fluid administration according to an embodiment.

FIGS. 5B and 5C depict cut-away views of an illustrative cap assembly having a spike in communication with a container for fluid administration according to an embodiment.

### DETAILED DESCRIPTION

The present disclosure discloses hollow spikes for use with a small-volume glass vial or any other fluid container to reduce the amount of waste fluid retained in the container after fluid administration.

FIG. 1 depicts a first illustrative spike for fluid administration according to an embodiment. As shown in FIG. 1, a spike 100 may include a hollow access tube 110, a platform 130, and a sharp tip-end 160. The hollow access tube 110 may extend from a proximal end of the spike 100 to a distal end of the spike. The platform 130 may surround the hollow access tube at a location between the distal end and the proximal end of the spike 100. The sharp tip-end 160 may be located at the distal end of the spike 100.

The hollow access tube 110 may include a central bore and a sidewall that each extend from the proximal end to the sharp-tip end 160. The central bore may be configured to enable a fluid to pass therethrough when the spike 100 is inserted into a fluid container (as further described herein). In an embodiment, the sharp-tip end 160 may permit a fluid to pass therethrough to the central bore of the hollow access tube 140, 110. The sidewall may be configured to surround the central bore and be substantially solid (except as described herein). The portion (i.e., 140) of the hollow access tube 110 that is distal from the platform 130 may include at least one opening, such as 150a-f. Each opening 150a-f may extend from the central bore to the sidewall. As such, fluid may flow via at least one of the one or more openings 150a-f to the central bore of the hollow access tube 140, 110.

In the embodiment shown in FIG. 1, the at least one opening comprises a plurality of holes 150a-f arranged in a column. In an embodiment, at least one opening 150a-f may extend radially from the central bore to the sidewall. In an embodiment, at least one opening 150a-f may extend in a non-radial manner from the central bore to the sidewall.

In an alternate embodiment depicted in FIG. 2, the spike 200 may include at least one opening that comprises a channel 210. The channel 210 may extend along at least a portion of the access tube 140 between the platform 130 and the sharp tip-end 160 and may extend from the central bore to the sidewall to enable fluid to pass therethrough. In an embodiment, the channel may be linear, helical, diagonal or any other shape. In an embodiment, a plurality of channels 210 may be fabricated in the spike 200.

In yet another embodiment depicted in FIG. 3, the spike 300 may include at least one opening comprising a plurality of holes 310a-k. At least a portion of a first hole, such as 310a, may overlap with at least a portion of a second hole, such as 310b, in a cross-sectional plane. In this manner, if the spike 300 is disposed in a vertical direction, at least one of the plurality of holes 310a-k may be only partially covered by a fluid in a container in which the spike 300 is disposed. In this way, the meniscus of the fluid may always be in contact with at least one opening 310a-k after the meniscus is in contact with the most distal opening until the meniscus of the fluid is below the most proximal opening. In an embodiment, the plurality of holes 310a-k may be arranged in a plurality of columns. In an embodiment, the plurality of holes 310a-k may be configured in an alternate arrangement, such as randomly, in a spiral, helical or diagonal pattern, or the like.

In an embodiment, the spike may further include a vent tube 120. The vent tube 120 may be configured to equalize a pressure in a container in which the spike, such as 100, is inserted as fluid is withdrawn from the container. In an embodiment, the vent tube 120 may include an air filter 125 to filter and/or sterilize the air that equalizes the pressure within the container. In an embodiment, the air filter 125 may be hydrophobic so that air may pass through, but fluid will not exit the vent tube 120.

In an embodiment, a tube (not shown) may be attached to the proximal end of the spike, such as 100. The tube may be configured to connect to the hollow access tube 110. The tube may be configured to deliver fluid from the hollow access tube 110 to a fluid administration location, such as a needle or other injection device inserted into a patient. In an embodiment, the tube may be flexible.

The spike embodiments 100-300 disclosed above may each enable fluid to flow through not only the sharp tip-end 160 of the spike, but also through the one or more openings, such as 150a-f, 210, or 310a-k. In this manner, fluid may be withdrawn from a container into which a spike designed according to the teachings of the present disclosure is inserted until the meniscus of the fluid is below the most proximal opening, instead of the sharp tip-end. As such, less fluid will remain in a container into which a spike designed according to the teachings of the present disclosure is inserted than if the fluid could only flow through the tip-end.

FIG. 4 depicts an illustrative fluid administration system according to an embodiment. As shown in FIG. 4, a fluid administration system may include a spike 410 and a fluid container 480. The spike may include a hollow access tube 420, a platform 440, and a sharp tip-end 470. The hollow access tube 420 may extend from a proximal end of the spike 410 to a distal end of the spike. The platform 440 may surround the hollow access tube at a location between the distal end and the proximal end of the spike 410. The sharp tip-end 470 may be located at the distal end of the spike 410.

The hollow access tube 420 may include a central bore and a sidewall that each extend from the proximal end to the sharp-tip end 470. The central bore may be configured to enable a fluid, such as 490, to pass therethrough when the spike 410 is inserted in a fluid container 480. In an embodiment, the sharp-tip end 470 may permit the fluid 490 to pass therethrough to the central bore of the hollow access tube 450, 420. The sidewall may be configured to surround the central bore and be substantially solid (except as described below). The portion (i.e., 450) of the hollow access tube 420 that is distal from the platform 440 may include at least one opening, such as 460a-d. Each opening 460a-d may extend from the central bore to the sidewall. As such, fluid may flow via at least one of the one or more openings 460a-d to the central bore of the hollow access tube 450, 420.

In the embodiment shown in FIG. 4, the at least one opening comprises a plurality of holes 460a-d arranged in a column. In an embodiment, the at least one opening 460a-d may extend radially from the central bore to the sidewall. In an embodiment, the at least one opening 460a-d may extend in a non-radial manner from the central bore to the sidewall. In embodiments, the spike may alternately include the spike 100 disclosed in FIG. 1, the spike 200 disclosed in FIG. 2, the spike 300 disclosed in FIG. 3 or any other spike embodying the teachings of this disclosure.

In an embodiment, the spike may further include a vent tube 430. The vent tube 430 may be configured to equalize a pressure in a container in which the spike 410 is inserted as fluid is withdrawn from the container. In an embodiment, the vent tube 430 may include an air filter 435 to filter and/or sterilize the air that equalizes the pressure within the container. In an embodiment, the air filter 435 may be hydrophobic so that air may pass through, but fluid will not exit the vent tube 430.

The fluid container 480 may be sealed with a septum 485. The sharp end-tip 470 may be inserted through the septum 485 and pushed until the platform 440 contacts the septum. In this configuration, it may be understood that at least a portion of the distal end 450 of the hollow access tube 420 may extend into the container 480 beyond the interior surface of the septum 485. Fluid 490 within the container 480 may be withdrawn through the distal end 450 of the hollow access tube 420 via either the sharp tip-end 470 or the one or more openings 460a-d. At least one of the one or more openings 460a-d may be between the sharp tip-end 470 and the septum 485. As such, the one or more openings 460a-d may permit fluid flow when the meniscus of the fluid 490 falls below the sharp tip-end 470. Indeed, the one or more openings 460a-d may enable fluid 490 to drain from the container 480 until the meniscus is below the most proximal exposed opening. As a result, less fluid 490 may remain in the container 480 than if the fluid only flowed through the sharp tip-end 470.

In an embodiment, the septum 485 may be tapered towards the distal end 450 of the hollow access tube 420 to enable substantially all of the fluid within the container 480 to be withdrawn. For example, the septum 485 may be tapered outward towards the distal end of the septum. In other words, the septum 485 may be tapered inward towards the proximal end of the septum at a location where the septum is configured to be pierced by the distal end 450 of the hollow access tube 420. In this manner, fluid may be funneled by the septum 485 towards the one or more openings 460a-d in the distal end 450 of the hollow access tube 420 when inserted through the septum.

In an embodiment, a tube (not shown) may be attached to the proximal end of the spike 410. The tube may be configured to connect to the hollow access tube 420. The tube may be configured to deliver the fluid 490 from the hollow access tube 420 to a fluid administration location, such as a needle or other injection device inserted into a patient. In an embodiment, the tube may be flexible.

In an embodiment, a spike according to an embodiment may be used in a gravity-fed fluid administration system. In an embodiment, a spike according to an embodiment may be used in a reduced pressure delivery system, such as drawing a fluid from a container in to a syringe.

FIG. 5A depicts an illustrative spike for fluid administration according to an embodiment. As shown in FIG. 5A, a spike 505 may be incorporated into a cap assembly 500. The spike 505 may include one or more channels, such as 510, extending from the base of the spike (i.e., from the cap assembly 500) towards the distal end of the spike. Alternate and/or additional openings in the spike 505 may also be used within the scope of this disclosure.

The spike 505 may be located substantially at a center of the cap assembly 500. In an embodiment, the cap assembly 500 may include a threaded inner surface 515 surrounding the spike 505. The threaded inner surface 515 may be used to affix the cap assembly 500 to a container having a threaded portion surrounding a septum. When attached to a container, the spike 505 may pierce a septum of the container, and the cap assembly 500 may be engaged via the threads with a threaded portion of the container. Indeed, the mechanical advantage of the threading action of the cap assembly 500 may assist in piercing the septum of a container. Moreover, the cap assembly 500 may be prevented from rotating and/or moving axially when the spike 505 is installed. Other methods of affixing a cap assembly 500 to a container are also considered within the scope of this disclosure and will be apparent to those of ordinary skill in the art.

In an embodiment, the cap assembly 500 may be used to removably attach and/or secure the spike 505. The cap assembly 500 may be particularly useful for a high pressure administration process (i.e., where the fluid container has a pressure that is above atmospheric pressure). In addition, the cap assembly 500 may be used for a gravity-fed administration process to ensure that the spike 505 does not separate from the container during administration of the fluid. The cap assembly 500 may be used for alternate or additional administration processes within the scope of this disclosure.

FIGS. 5B and 5C depict the cap assembly of FIG. 5A in communication with a container according to an embodiment. As shown in FIGS. 5B and 5C, when the cap assembly 500 is inserted into the container 520, the spike 505 may pierce a septum 525 of the container. In an embodiment, the cap assembly 500 may include a sealing surface 530 that is located proximal to the septum 525 of the container 520 when the spike 505 is inserted into the container. In an embodiment, the sealing surface 530 may surround the central portion of the spike 505 at a location between the distal end and the proximal end of the spike.

In an embodiment, the sealing surface 530 may be tapered such that the sealing surface at the portion surrounding the spike may be proximal with respect to the edges of the sealing surface. In such an embodiment, pressurized gas (for a high pressure administration system) and/or fluid is directed towards the spike 505 across the septum 525 and against the sealing surface 530 so that the pressurized gas or fluid may not be trapped between the septum 525 and the sealing surface 530.

In an embodiment, the spike 505 may allow fluid to be in fluid communication with the septum 525 and the sealing surface 530. In other words, the spike 505 may be configured to fluidly connect the fluid container 520 to a space between the septum 525 and the sealing surface 530 when the spike is inserted through the septum. In an embodiment, the one or more channels 510 of the spike may be located on both a portion of the spike 505 that is distal of the septum 525 and a portion of the spike that is proximal of the septum, but distal of the sealing surface 530 when the spike is inserted into the fluid container 520. In an alternate embodiment, the one or more channels 510 may extend from a portion of the spike 505 that is distal of the septum 525 to a portion of the spike that is proximal of the septum, but distal of the sealing surface 530 when the spike is inserted into the fluid container 520.

During administration, the fluid may pass through the channel in the spike 505 to a delivery tube 535.

In an embodiment, an O-ring may be used to form a seal in lieu of or in addition to the sealing surface 530 of FIGS. 5B and 5C. In an alternate embodiment, a gasket type seal may be used to form a seal in lieu of or in addition to the sealing surface 530 of FIGS. 5B and 5C. Alternate and/or additional sealing mechanisms known to those of ordinary skill in the relevant art may also be used within the scope of this disclosure.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

What is claimed is:

1. A spike for fluid administration, the spike comprising:
a hollow access tube extending from a proximal end of the spike to a distal end of the spike;
a platform surrounding the hollow access tube at a location between the distal end and the proximal end; and
a sharp tip-end located at the distal end of the spike,
wherein the hollow access tube has a central bore and a sidewall, each extending from the proximal end to the sharp tip-end,
wherein the hollow access tube comprises at least one opening extending from the central bore to the sidewall and located between the platform and the sharp tip-end.

2. The spike of claim 1, wherein the at least one opening comprises one or more holes.

3. The spike of claim 2, wherein the one or more holes comprise a plurality of holes, wherein at least a portion of a first hole overlaps with at least a portion of a second hole in a cross-sectional plane.

4. The spike of claim 2, wherein the one or more holes comprise a plurality of holes in a plurality of columns, wherein at least a portion of each hole in a first column of the plurality of columns overlaps with at least a portion of a hole in a second column of the plurality of columns in a cross-sectional plane.

5. The spike of claim 1, wherein the at least one opening comprises one or more channels.

6. The spike of claim 1, further comprising:
a vent tube having an air filter, wherein the vent tube is configured to equalize a pressure in a container in which the spike is inserted as fluid is withdrawn from the container.

7. The spike of claim 1, further comprising a cap assembly configured to removably attach the spike to a container.

8. A spike and tubing set for fluid administration, the set comprising:
a spike comprising:
a hollow access tube extending from a proximal end of the spike to a distal end of the spike,
a platform surrounding the hollow access tube at a location between the distal end and the proximal end, and
a sharp tip-end located at the distal end of the spike,
wherein the hollow access tube has a central bore and a sidewall, each extending from the proximal end to the sharp tip-end,
wherein the hollow access tube comprises at least one opening extending from the central bore to the sidewall and located between the platform and the sharp tip-end; and
a flexible tube configured to connect to the hollow access tube and to deliver fluid from the hollow access tube to a fluid administration location.

9. The spike and tubing set of claim 8, wherein the at least one opening comprises one or more holes.

10. The spike and tubing set of claim 9, wherein the one or more holes comprise a plurality of holes, wherein at least a portion of a first hole overlaps with at least a portion of a second hole in a cross-sectional plane.

11. The spike and tubing set of claim 9, wherein the one or more holes comprise a plurality of holes in a plurality of columns, wherein at least a portion of each hole in a first column of the plurality of columns overlaps with at least a portion of a hole in a second column of the plurality of columns in a cross-sectional plane.

12. The spike and tubing set of claim 8, wherein the at least one opening comprises one or more channels extending axially towards the proximal end of the spike and the distal end of the spike.

13. The spike and tubing set of claim 8, wherein the spike further comprises a vent tube having an air filter, wherein the vent tube is configured to equalize a pressure in a container in which the spike is inserted as fluid is withdrawn from the container.

14. A fluid administration system comprising:
a fluid container having a septum and containing a fluid; and
a spike comprising:
a hollow access tube extending from a proximal end of the spike to a distal end of the spike,
a sealing surface surrounding the hollow access tube at a location between the distal end and the proximal end, and
a sharp tip-end located at the distal end of the spike,
wherein the hollow access tube has a central bore and a sidewall, each extending from the proximal end to the sharp tip-end,
wherein the hollow access tube comprises at least one opening extending from the central bore to the sidewall and located between the sealing surface and the sharp tip-end.

15. The fluid administration system of claim 14, further comprising:
a flexible tube configured to connect to the hollow access tube and to deliver fluid from the hollow access tube to a fluid administration location.

16. The fluid administration system of claim 14, wherein the at least one opening comprises one or more holes.

17. The fluid administration system of claim 16, wherein the one or more holes comprise a plurality of holes, wherein at least a portion of a first hole overlaps with at least a portion of a second hole in a cross-sectional plane.

18. The fluid administration system of claim 16, wherein the one or more holes comprise a plurality of holes in a plurality of columns, wherein at least a portion of each hole in a first column of the plurality of columns overlaps with at least a portion of a hole in a second column of the plurality of columns in a cross-sectional plane.

19. The fluid administration system of claim 14, wherein the at least one opening comprises one or more channels extending axially towards the proximal end of the spike and the distal end of the spike.

20. The fluid administration system of claim 14, wherein the spike further comprises a vent tube having an air filter, wherein the vent tube is configured to equalize a pressure in the fluid container as fluid is withdrawn from the fluid container.

21. The fluid administration system of claim 14, wherein the spike is configured to be inserted into the fluid container through the septum, wherein each of the at least one openings is configured to be distal from the septum upon insertion of the spike into the fluid container.

22. The fluid administration system of claim 14, wherein the sealing surface is tapered from the edges towards the hollow access tube such that the sealing surface is configured to direct the fluid towards the hollow access tube when the fluid is withdrawn from the fluid container.

23. The fluid administration system of claim 14, wherein the fluid administration system is a gravity fed fluid administration system.

24. The fluid administration system of claim 14, wherein the fluid container is pressurized at a pressure above atmospheric pressure.

25. The fluid administration system of claim 14, wherein the spike is configured to fluidly connect the fluid container to a space between the septum and the sealing surface in response to the spike being inserted through the septum.
